(19) United States
(12) Patent Application Publication
(10) Pub. No.: US 2020/0282120 A1
(43) Pub. Date: Sep. 10, 2020

(54) METHOD OF IDENTIFYING MOTOR OF MEDICAL PUMP, METHOD OF DRIVING MOTOR OF MEDICAL PUMP, CONTROLLER, AND VENTRICULAR ASSIST SYSTEM

(71) Applicant: SUN MEDICAL TECHNOLOGY RESEARCH CORPORATION, Nagano (JP)

(72) Inventor: Masahiko ITO, Nagano (JP)

(21) Appl. No.: 16/807,162

(22) Filed: Mar. 3, 2020

(30) Foreign Application Priority Data
Mar. 6, 2019 (JP) 2019-040726

(51) Int. Cl. A61M 1/10; H02K 5/132

(52) U.S. Cl. CPC A61M 1/1086; A61M 2205/3334; H02K 5/132; A61M 1/101

(57) ABSTRACT

A blood pump (medical pump) has a three-phase Y-connection motor formed of coils of three phases consisting of a U phase coil, a V phase coil and a W phase coil. Using a blood pump controller (controller), a current value of an electric current between the coils of two phases is detected by applying a direct current voltage or an alternating current voltage to the coils of any two phases among the coils of the three phases of the motor which is an object to be driven, and the motor which forms an object to be driven is identified by determining whether the current value which is detected is more than a threshold value which is preliminarily set or equal to or less than the threshold value.

### BACKGROUND OF THE INVENTION

### 1. Field of the Invention

The present invention relates to a method of identifying a motor of a medical pump, a method of driving a motor of a medical pump, a controller, and a ventricular assist system.

### 2. Description of the Related Art

Conventionally, as a medical pump, there has been known a blood pump used in a ventricular assist system or a vacuum pump used in a medical aspirator. The ventricular assist system is formed of: a blood pump which is embedded and retained in a living body; and a controller which controls the blood pump outside the living body (referred to as “blood pump controller”) The medical aspirator is formed of a vacuum pump for suction, a motor which drives the vacuum pump, and a controller which controls the motor (referred to as “control unit”).

The blood pump must be properly controlled by the blood pump controller and hence, usually, a dedicated-use blood pump controller which corresponds to a blood pump which is an object to be retained in a living body is used. When plural kinds of blood pumps exist, controllers corresponding to the respective blood pumps are selectively used. Accordingly, it is necessary to prepare a plurality of blood pump controllers corresponding to kinds of the blood pumps. Which blood pump is used in which patient cannot be directly confirmed since the blood pump is retained in the living body. Of course, although the identification can be made by referencing a medical record, there is a concern that a human error such as misreading or mis-recording occurs.

On the other hand, in a medical aspirator, after a vacuum pump unit is used, the vacuum pump unit is removed from the motor unit and is discarded. When the medical aspirator is used next time, during an in-use time, an unused vacuum pump unit is mounted on the motor unit. When plural kinds of motors exist, it is necessary to select the vacuum pump unit which conforms to a motor specification and to mount the vacuum pump unit on the motor unit. Also in the medical aspirator, it is difficult to directly visually recognize the motor on a medical site and hence, it is not deniable that a human error occurs relating to the selection of the motor at the time of mounting the vacuum pump unit on the motor unit.

Since the blood pump and the vacuum pump are driven by the motor, if the motor can be identified at the time of driving the motor, it is possible to prevent a human error at the time of mounting the motor on the blood pump and the vacuum pump. JP 2017-123729 A discloses a method of identifying a motor.

In the method of identifying a motor disclosed in JP 2017-123729 A, a motor identification signal line is connected to one or two coils among a U phase coil, a V phase coil, and a W phase coil, and winding specification is identified by applying a voltage pulse to the coil. That is, a voltage pulse is applied to any one of three-phase coils, the motor is identified based on a change of the motor identification signal, and a control parameter which matches the identified motor is selected from a table prepared in advance, and the motor is driven based on the selected control parameter.

### SUMMARY OF INVENTION

However, the method of identifying a motor described in JP 2017-123729 A has a drawback that the identification of the motor can be only possible with respect to the motor where the motor identification signal line is connected to either one or two coils among three-phase coils. Further, the control parameter is set, the winding specification of the coil is selected from the table of the control parameter based on the identified motor (winding specification) and, thereafter, the motor is driven using the selected control parameter. Accordingly, there is a concern that a human error occurs in preparing the control parameter and the table. In the medical pump, it is a prerequisite that a motor which cannot be directly visually recognized is identified with certainty and is driven under a proper drive condition.

The present invention has been made to overcome such drawbacks, in a state where plural kinds of medical pumps exist, it is an object of the present invention to realize a method of identifying a motor of a medical pump which can identify a motor within a short time without connecting a motor identification signal line, a method of driving a motor of a medical pump which can eliminate a human error from identification of a motor to steady-state driving of the motor, and the identification of a motor among plural kinds of medical pumps using one controller. Further, it is an object of the present invention to realize a controller and a ventricular assist system which can eliminate a human error from starting of a motor to steady state driving of the motor.

[1] A method of identifying a motor of a medical pump according to the present invention is a method of identifying a motor of a medical pump, the motor of the medical pump being a motor of a three-phase Y-connection formed of coils of three phases consisting of a U phase coil, a V phase coil and a W phase coil, the method comprising the steps of: detecting a current value of an electric current between the coils of two phases by applying a direct current voltage or an alternating current voltage to the coils of any two phases among the coils of the three phases of the motor which is an object to be driven; and identifying the motor which is the object to be driven by determining whether the current value which is detected is more than a threshold value which is preliminarily set or equal to or less than the threshold value.

For example, in the ventricular assist system, the pump (hereinafter referred to as blood pump) is embedded and retained in a living body, and a drive control of the pump is performed by a controller disposed outside the living body. Usually, there is almost no case where the blood pump itself is exchanged, there is a reasonable number of chances that the controller which a person operates or touches is exchanged. When the controller is exchanged, it is necessary to exchange with a controller capable of driving the motor with a control parameter which matches the specification of a motor which is integrally formed with the retained blood pump. However, when plural kinds of the blood pumps exist, it is impossible to identify the motor retained in the living body by directly visually recognizing the motor. For example, also in a medical aspirator, it is difficult to identify a motor stored in a device by directly visually recognizing the motor.

According to the method of identifying a motor of a medical pump of the present invention, a current value of an electric current which flows between two phases is measured by applying a voltage pulse to the coils of two-phases, that is, the U phase and the V phase, for example, among the coils of three-phases, the measured current value is compared with a preset threshold value, and the motor can be identified based on whether or not the measured current value is larger or smaller than the threshold value. Identifying the motor which is integrally formed with the medical pump is equal to identifying the medical pump. According to such an identifying method, with respect to the motors of the plural kinds of medical pumps existing in the living body which are difficult to directly visually recognize, unlike the prior art, it is possible to identify the motor within a short time without connecting a motor identification signal line to the motor. Accordingly, it is possible to eliminate a human error in the motor identifying step.

[2] In the method of identifying a motor of a medical pump according the present invention, it is preferable that detection of the current value is intermittently performed plural times within a predetermined time, and the motor which is the object to be driven is identified by determining whether all measured current values are more than the threshold value or equal to or less than the threshold value.

For example, the current measurement is performed 9 times per 1 second, and it is determined whether or not all measured current values are more than the threshold value or equal to or less than the threshold value. In this manner, by performing the current measurement plural times intermittently per the predetermined time and by comparing the measured current value with the threshold value each time, the motor can be identified within a short time, and the reliability of the motor identification can be enhanced.

[3] In the method of identifying a motor of a medical pump according the present invention, it is preferable that the threshold value be set by taking into account irregularities of a current value attributed to an influence of a coil impedance which is an object to be measured or a surface temperature of the motor during driving.

When the medical pump is driven, that is, when the motor is driven, there may be a case where the motor generates heat so that a temperature of the motor is increased. Further, the medical pump which is retained in a human body is also influenced by a temperature of the human body. A resistance value of the coil changes corresponding to a change in the temperature and a current value changes corresponding to the change in the resistance value of the coil. Accordingly, by setting a threshold value while taking into account a change in coil impedance brought about by a change in temperature, it is possible to realize identification of the motor with high reliability which conforms to actual driving of the motor.

[4] In the method of identifying a motor of a medical pump according the present invention, it is preferable that a control parameter which matches the motor which is identified among a plurality of the control parameters be selected.

In the above-mentioned method, the control parameter includes, for example, the number of magnetic poles, coil impedance, inductance and the like which are main factors relating to the motor specification. Accordingly, by selecting the control parameter of the motor which is the object to be driven among the control parameters for each motor, the drive condition of the motor can be decided and hence, the occurrence of a human error in the steps ranging from the identification of the motor to the motor driving can be eliminated.

[5] A method of driving a motor of a medical pump according to the present invention includes the steps of: identifying the motor which is an object to be driven by the method of identifying a motor of a medical pump according to any one of claims 1 to 3: selecting a control parameter which matches the identified motor: performing magnetic pole alignment between a rotor and a stator by applying a voltage to the motor for a predetermined time: constantly increasing a rotational speed of the motor by applying a motor start pulse to the motor for a predetermined time: and driving the motor at a rotational speed of a steady-state driving of the medical pump, wherein the steps are autonomously switched in accordance with a sequence programmed in the controller.

In such a method of driving a motor of a medical pump, the step of identifying the motor, the step of selecting the control parameter, the step of performing magnetic pole alignment, and the step of constantly increasing a rotational speed of the motor to a rotational speed for steady-state driving are automatically sequentially switched in accordance with a sequence. Accordingly, a human error which may occur from the identification of the motor to the steady-state driving can be eliminated. The steady-state driving is a state where the motor is driven at a rotational speed set at the time of operating the medical pump in a steady state.

[6] In the method of driving a motor of a medical pump according to the present invention, it is preferable that a drive control of the motor be performed by a PWM control, and a voltage pulse applied to the motor be switched to a duty of the voltage pulse at the magnetic pole alignment, a duty of the motor start voltage pulse and a duty of a steady-state driving voltage pulse sequentially after a lapse of a predetermined time.

In starting driving of the motor, the motor is controlled such that the step-out does not occur at the time of starting driving of the motor by making the magnetic pole position of the rotor and the magnetic pole position of the stator (coils) aligned with each other. The motor is not rotated in magnetic pole position alignment. Further, since a rotational load of the motor at the time of starting driving of the motor (at the time of starting the rotation of the motor) is large, a drive torque is increased. The drive torque is set such that stable rotation can be achieved in steady-state driving. In this manner, by setting appropriate duties at the respective steps of magnetic pole position alignment, motor starting and steady-state driving of the motor, the medical pump can be stably operated within a short time. For example, the blood pump which is retained in the living body is required to be brought into a stable drive state within a short time.

[7] A controller according to the present invention is a controller for controlling the motor of a medical pump, the controller including: a switching circuit part configured to apply a direct current voltage or an alternating current voltage in a accordance with a predetermined order to the respective coils of the three phases; a control part configured to control a current detection circuit part configured to measure an electric current which flows into coils of any two phases among the coils of the three phases; a current comparison determination part configured to determine the motor which is the object to be driven by comparing a measured current value with a threshold value, and a control parameter selection part configured to select a control parameter which matches the motor which is the object to be driven among a plurality of the control parameters preliminary set based on the measured current value.

The controller identifies the motor which is the object to be driven based on the detected current value, selects the control parameter which matches the motor, and drives the motor. The control part performs a control of the entirety of the medical pump and the controller. The switching circuit part has a function of applying a voltage to either one of or all of the U phase, the V phase and the W phase in set order based on the control parameter, and a function of inputting a motor drive signal to the motor. By autonomously sequentially switching the steps ranging from the identification of the motor to the steady-state driving, a human determination action does not exist and hence, a human error can be eliminated. Further, by preparing the control parameters which correspond to plural kinds of motors in the controller, the plural kinds of motors can be identified using one controller, and the controller can drive the motor which is the object to be driven using the control parameter which matches the motor which is the selected object to be driven.

[8] A ventricular assist system according to the present invention includes: a medical pump embedded and retained in a living body: and the controller described in [7] disposed outside the living body and connected to the medical pump through a medical tube.

The ventricular assist system according to the present invention has the controller described in the above-mentioned [7]. According to the ventricular assist system having such a configuration, by connecting the blood pump controller to the blood pump and by starting the blood pump, the steps ranging from the identification of the motor, that is, the blood pump retained in the living body to the driving in a steady state can be performed autonomously without requiring human operations and human determinations. As a result, according to the ventricular assist system of the present invention, it is possible to prevent the occurrence of a human error and hence, the ventricular assist system can be used safely. For example, an electric signal line which connects the controller and the pump to each other and the like pass through the medical tube.

### BRIEF DESCRIPTION OF DRAWINGS

FIG. 1 is a block diagram showing the system configuration of a blood pump controller;

FIG. 2 is a flowchart showing main steps of method of driving a blood pump;

FIG. 3 is a graph schematically showing a consumption current and a rotational speed which change along with a lapse of time after starting the blood pump;

FIG. 4 is a view for describing one example of a relationship between the distribution of measured current values and a threshold value; and

FIG. 5 is an explanatory view showing one example of a ventricular assist system.

### DESCRIPTION OF PREFERRED EMBODIMENTS

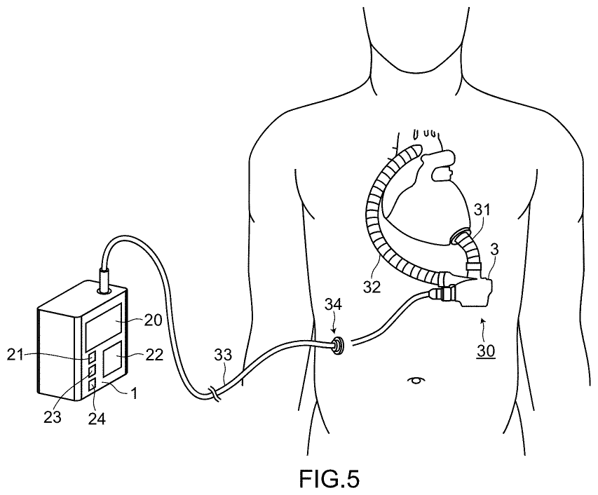

In an embodiment described hereinafter, a case is described where a blood pump 3 is used as a medical pump in a ventricular assist system 30 (see FIG. 5) and is retained in a living body as an example. Also in this case, a blood pump controller 1 is used as a controller, and a blood pump control part 6 is used as a control part.

### [Configuration of Blood Pump Controller 1]

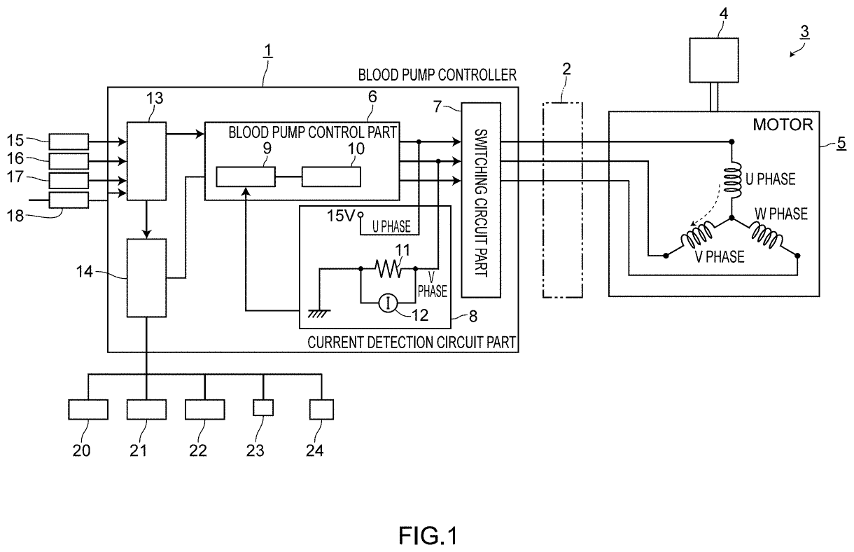

FIG. 1 is a block diagram showing the system configuration of the blood pump controller 1. The blood pump 3 is connected to the blood pump controller 1 via a connector 2. The blood pump 3 includes a pump portion 4 and a motor 5 which rotates an impeller (not shown in the drawing) of the pump portion 4. In the description made hereinafter, plural kinds of existing motors are collectively referred to as motor 5. The motor 5 is a DC brushless motor, and is formed of coils of three-phase y connection and a rotor made of a permanent magnet (not shown in the drawing). The coils of three phases include a U phase coil, a V phase coil and a W phase coil. In the description made hereinafter, these respective coils may be also referred to as a U phase, a V phase and a W phase.

The blood pump controller 1 includes: a blood pump control part 6 which performs drive control of the blood pump 3 (that is, the motor 5) in accordance with a sensorless vector control; a switching circuit part 7 configured to input a voltage to any one of or all of the U phase, the V phase and the W phase in set order in response to an output signal from the blood pump control part 6; and a current detection circuit part 8. The blood pump control part 6 has a current comparison determination part 9 and a control parameter selection part 10 incorporated in a software. The blood pump control part 6 is a microcomputer (CPU) which controls a drive control of the blood pump 3 and the whole blood pump controller 1. The current detection circuit part 8 is formed of a shunt resistor 11, and an ammeter 12 connected to the shunt resistor 11 in parallel. In the example shown FIG. 1, the ammeter 12 is provided for measuring an electric current which flows from the U phase to the V phase, wherein a voltage of 15V is applied to the U phase, and the V phase is grounded via the shunt resistor 11 and the ammeter 12. That is, an electric current flows from the U phase to the V phase (indicated by a dotted line in FIG. 1).

However, a case may be considered where an electric current which flows from the V phase to the W phase may be measured by applying a voltage of 15V to the V phase and connecting the W phase to a ground. Alternatively, an electric current which flows from the W phase to the U phase may be measured by applying a voltage of 15V to the W phase and by connecting the U phase to a ground. All such configurations may be arbitrarily set in the software incorporated in the blood pump control part 6. A voltage of 15V applied to the coils is one example and is not limited.

The current comparison determination part 9 has a function of determining which prescribed threshold value range a current value belongs by comparing a current valued measured by the current detection circuit part 8 and a threshold value preliminary incorporated in a memory part of the blood pump control part 6. The threshold value is a value which is preliminarily set corresponding to the blood pump 5 which is an object to be used. That is, the current comparison determination part 9 identifies a specification of the motor 5 used in the blood pump 3 retained in a living body.

The control parameter selection part 10 selects a control parameter corresponding to the motor 5 identified by the current comparison determination part 9, and inputs a drive signal to the switching circuit part 7. Although the control parameter includes many items, as main factors relating to the motor specification, the number of magnetic poles, coil impedance and inductance are named. Further, the control parameter also contains a plurality of parameters associated with these parameters. An applying voltage and a frequency of a voltage pulse corresponding to an object to be driven are decided based on the selected control parameter, the motor 5 is driven. The switching circuit part 7 inputs a motor drive signal to the motor 5 by sequentially applying a voltage to any one of or all of the U phase, the V phase and the W phase based on a command from the blood pump control part 6. The control parameter is stored in the memory part of the software of the blood pump control part 6.

The blood pump controller 1 further includes a power source control part 13 and a user interface part 14. The blood pump controller 1 has power sources from four paths. In an example shown in FIG. 1, as the power sources from four paths, the blood pump controller 1 has a first battery 15, a second battery 16, an emergency battery 17, and a commercially available power source input part 18. Since the blood pump controller 1 is configured to allow a user (patient) to carry with him/her even when the user moves, a main power source is formed of a battery. The second battery 16 complements the first battery 15. Further, the blood pump controller 1 can use a commercially available power source. The emergency battery 17 is automatically chosen by switching by the power source control part 13 when abnormality is recognized in the second battery 16 and the commercially available power source. The power source control part 13 has a function of controlling electricity inputted from the above-mentioned respective power sources to an appropriate voltage, a function of converting electricity from the commercially available power source to direct current electricity and the like. Alternating current electricity from the commercially available power source may be converted into direct current electricity via a RLC serial circuit.

The user interface part 14 controls a display part 20, a lamp 21, an input part 22, a buzzer 23 and a main switch 24. The display part 20 is a liquid crystal display, an organic EL display or the like, and displays set information such as a drive condition of the blood pump 3, drive information, user information and the like. The lamp 21 and the buzzer 23 notify a user the occurrence of abnormality when a state where the blood pump 3 is abnormally driven is detected. Abnormal driving means lowering of a voltage of the battery, stepping out of the motor 5, detection of an over current or the like. The input part 22 has a function of inputting a user name, set information and the like. The display part 20 may be formed as an input part in the form of a touch panel, and the display part 20 may be also used as the input part together with the input part 22. The main switch 24 has a function of starting-stopping (ON/OFF) of the blood pump controller 1.

Although not shown in the drawings, the blood pump controller 1 includes; a feedback part which constantly detects a rotational speed of the motor 5, and feedbacks the rotational speed to the blood pump control part 6; and an overcurrent stop circuit for stopping the supply of an overcurrent to the motor 5 and the like. Further, the blood pump controller 1 may be connected to an external monitor by which a medical staffs such as doctors or nurses can monitor a drive state of the blood pump 3.

The blood pump controller 1 described above is a device which controls the blood pump 3 used in the ventricular assist system 30 (see FIG. 5). The blood pump controller 1 includes; a switching circuit part 7 which applies a direct current voltage or an alternating current voltage to the coils of three phases (U phase, V phase, W phase) respectively in a predetermined order; a current detection circuit part 8 which measures an electric current which flows in the coils of any two phases among coils of three phases (U phase, V phase, W phase); and a blood pump control part 6 which controls a current comparison determination part 9 which determines the motor is the motor 5 which is an object to be driven by comparing a measured current value and a threshold value and a control parameter selection part 10 which selects a control parameter corresponding to the motor 5 which is the object to be driven among a plurality of control parameters based on the measured current value.

With such a configuration, the motor 5 which is the object to be driven is identified based on the detected current value, and the motor 5 is driven by selecting the control parameter which matches the motor 5. Accordingly, the blood pump controller 1 can exclude a determination behavior of a person by autonomously and sequentially performing switching from identification of the motor 5 to steady-state driving and hence, a human error can be excluded. Further, by preparing control parameters which match the plurality of respective motors in the blood pump controller 1 in advance, it is possible to identify the plural kinds of the motors 5, that is, the blood pumps 3 using one blood pump controller 1, and it is possible to drive the blood pump 3 based on the control parameter which matches the motor 5 which is the object to be driven.

### [Method of Identifying Motor of Medical Pump and Method of Driving the Motor]

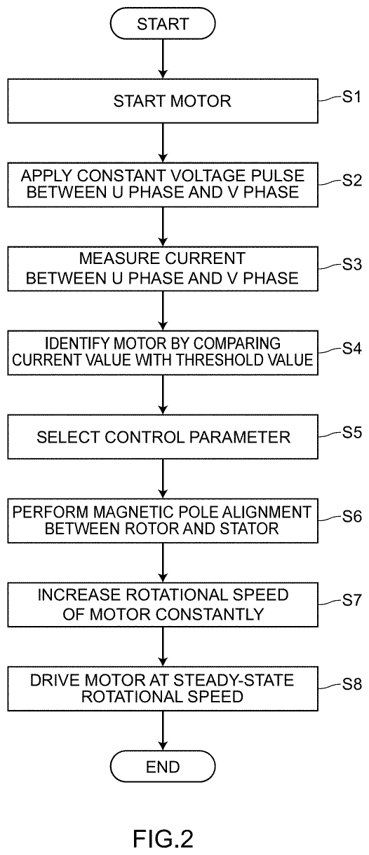

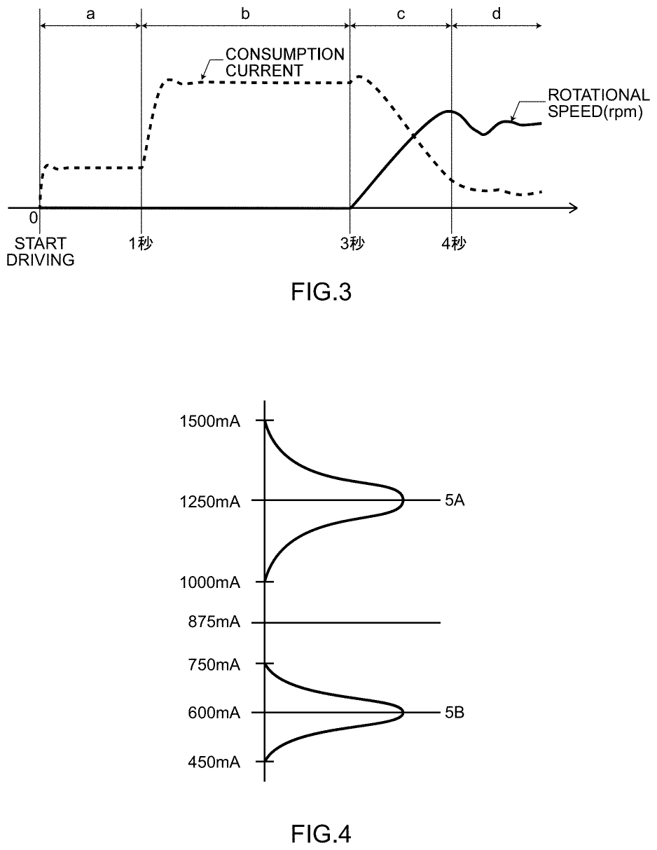

Subsequently, the method of identifying a motor and a method of driving the motor are described with reference to FIG. 2, FIG. 3 and FIG. 4 by taking the blood pump 3 as an example of a medical pump. The respective steps ranging from motor identification to steady-state driving are autonomously performed in accordance with a software and a sequence.

FIG. 2 is a flowchart showing main steps of the method of driving the blood pump 3. FIG. 3 is a graph schematically showing a consumed current and a rotational speed of the motor 5 which change in respective steps in accordance with a lapse of time from staring driving of the motor 5 (from turning on the main switch). FIG. 4 is a graph showing one example of a relationship between the distribution of measured current values and a threshold value. In FIG. 4, threshold values used for identification when two kinds of blood pumps 3 (that is, motors 5) are used. The steps are described in accordance with the flowchart shown in FIG. 2.

Firstly, the motor 5 is started by turning on the main switch 24 (step S1). In this embodiment, starting driving of the motor 5 has the same meaning as starting the blood pump controller 1. The blood pump control part 6 applies a voltage for current measurement between a U phase and a V phase via the switching circuit part 7 (step S2). Subsequently, the current detection circuit part 8 measures an electric current between the U phase and the V phase (step S3). In this embodiment, a width of pulse applying time (PWM) is calculated such that a pulse having a peak voltage of 15V and a frequency of 20 kHz is intermittently applied 9 times per 1 second so that a motor voltage becomes 5V. Then, a PWM signal is formed on a carrier and the PWM signal is outputted to the switching circuit 7, and an electric current between the U phase and the V phase is measured by the current detection circuit part 8 each time the PWM signal is outputted. Electricity is supplied only between the U phase and the V phase during a period that an electric current is measured and hence, the motor 5 is not rotated (see a region indicated by a in FIG. 3). Subsequently, the measured current value is compared with the threshold value so as to identify the motor 5 (step S4). The above-mentioned applied voltage and frequency are only one example and are not limited.

After the motor 5 is identified, a control parameter is selected (step S5). For example, in the case where two kinds of motors, that is, the motor 5A and the motor 5B are provided as the motors 5, when the motor 5A is identified, the control parameter which matches the motor 5A is selected. With respect to the control parameters, as main factors, the number of magnetic poles, coil impendence and inductance are named. Further, the control parameter further includes a plurality of parameters affiliated with these main factors. Subsequently, magnetic pole alignment between the rotor and the stator (coils) is performed by supplying an electric current to the U phase, the V phase and the W phase for a predetermined time (for example, 2 seconds) (step S6: see a region indicated by b in FIG. 3) In this embodiment, a signal of a PWM control which sets a current value of the motor 5A and a current value of the motor 5B at the same level is inputted to the switching circuit part 7. After a lapse of two seconds, a voltage pulse for driving the motor is applied to the motor 5A so that the motor 5A is rotated. In this embodiment, a motor starting torque is necessary and hence, a rotational speed of the motor 5A is increased at a predetermined rate (step S7: see a region indicated by c in FIG. 3). To be more specific, the rotational speed is gradually increased by defining a lead angle per unit time (also referred to as a forced commutation mode). At a point of time that the rotational speed of the motor 5A reaches a predetermined rotational speed, driving of the motor 5A is continued at a steady-state rotational speed (step S8: see a region indicated by d in FIG. 3)

Subsequently, motor identification, magnetic pole alignment, starting of the motor and the steady-state driving of the motor with respect to a time axis are described with reference to FIG. 3 by taking one example. A lapsed time (second) from starting of the motor (turning on the switch) is taken on an axis of abscissas. A time range in the region indicated by a is a region relating to motor identification based on current measurement, and is set to 1 second. A time range of the region indicated by b is a region for magnetic pole alignment between the rotor and the stator, and is set to 2 seconds. Within the range (a+b) for current measurement (motor identification) and magnetic pole alignment, the motor 5 is not rotated. Within a time range of the region indicated by c, a rotational speed of the motor 5 is gradually increased within 1 second (referred to as a starting operation), the motor enters a region of steady-state driving (predetermined rotational speed rpm) by finishing the starting operation of the motor after a lapse of 1 second, and this rotational speed is maintained until driving of the motor 5 is stopped thereafter (the region indicated by b).

As shown in FIG. 3, a consumed current after starting driving of the motor has a relationship of the consumed current in motor identification (the region indicated by a)<the consumed current in magnetic pole alignment (the region indicated by b). The motor 5 has the consumed current relationship relating to driving by a PWM control and hence, a duty ratio in the motor identification region is set smaller than the duty ratio in magnetic pole alignment. In starting driving of the motor, a rotational speed is increased at a predetermined rate, and the duty ratio is shifted to a duty ratio in steady-state driving when the rotational speed reaches a rotational speed for the steady-state driving.

A drive control of the motor 5 is performed by a PWM control and hence, power consumption for motor identification is smaller than power consumption for magnetic pole alignment. That is, a duty ratio in the region of motor identification is smaller than the duty ratio in the region for magnetic pole alignment. On the other hand, in the motor rotation starting region, the duty ratio is gradually shifted to the duty ratio in steady-state driving after starting driving of the motor and hence, a consumed current is gradually lowered, and it is possible to continue driving of the motor 5 at a duty ratio where power consumption is smallest until the motor 5 is stopped.

Next, a relationship between motor identification and a threshold value is described with reference to FIG. 4. FIG. 4 shows an example where two kinds of motors 5A, 5B exist. That is, FIG. 4 shows the example where a voltage pulse having a voltage peak of 15V and a frequency of 20 kHz is applied to the motors 5A, 5B. As one example, the current value distribution of the motor 5A having impedance (resistance value) of 3Ω is expressed on an upper portion of FIG. 4, and the current value distribution of the motor 5B having impedance of 6Ω is expressed on a lower portion of FIG. 4. In the example shown in FIG. 4, 875 mA is set as a threshold value. That is, when a measured value is more than 875 mA, the motor 5 is determined to be the motor 5A, while when the measured value is equal to or less than 875 mA, the motor 5 is determined to be motor 5B. In a state where both motors 5A, 5B are retained in a living body in a stationary manner, surface temperatures of the motors 5A, 5B are at a human body temperature level. On the other hand, when the blood pump 5 is driven, there may be a case where surface temperatures of the motors 5A, 5B are increased to approximately 40° C. In a case where the coil is made of copper, when a temperature of the coil is increased, impedance of the coil is increased so that a current value of the coil is lowered. When the temperature of the coil is lowered, the impedance of the coil is lowered so that the current value of the coil is increased.

In view of the above, in the motors 5A, 5B, the threshold value is set assuming a temperature change of from 0° C. to 120° C. by taking into account a tolerance. In the example of the current value distribution shown in FIG. 4, an impedance and the 50 distribution of a current value which takes into account an influence of a temperature change in advance are expressed. A current lower limit value of the motor 5A is 1000 mA and an upper limit value of the motor 5B is 750 mA and hence, it is preliminarily checked that the lower limit value of the motor 5A and the upper limit value of the motor 5B intersect with each other. Accordingly, it is understood that motor identification can be performed by measuring a current value of an electric current which flows from the U phase to the V phase.

In the method of identifying a motor of a medical pump described above, the blood pump 3 which is a medical pump has the motor 5 of a three-phase Y connection method formed of coils of three-phases consisting of the U phase coil, the V phase coil and the W phase coil. A current value between the coils of two phases (U phase-V phase) by applying a direct current voltage or an alternating current voltage to the coils of any two phases (U phase-V phase in this embodiment) among the coils of three-phases of the motor 5 which is an object to be driven is detected by the blood pump controller 1 which is the controller, and the motor 5 which is the object to be driven is identified by determining whether the detected current value is more than the threshold value which is preliminarily set or equal to or less than the threshold value.

According to such a method of identifying a medical pump, a current value of an electric current which flows between two-phases is measured by applying a direct current voltage or an alternating current voltage to the coils of two-phases that is, the U phase and the V phase among the coils of three-phases, the measured current value is compared with a preset threshold value, and the motor can be identified based on whether or not the measured current value is larger or smaller than the threshold value. In the example shown in FIG. 4, when the current value is larger than the threshold value (875 mA), the motor is identified as the motor 5A, while when the current value is smaller than the threshold value, the motor 5 is identified as the motor 5B. By adopting such a method, unlike the prior art, the motor can be identified within a short time without connecting motor identification signal lines to the motors. Further, unlike the prior art where the motor identification signal lines are connected to the motors, there is no possibility that the identification of the motor is affected by external noises or a wire resistance. The method of identifying a motor of a medical pump described above is applicable to the identification of a motor which drives a vacuum pump such as a medical aspirator.

In the method of identifying a motor of a medical pump, the detection of a current value of an electric current which flows between the U phase and the V phase is intermittently performed plural times within a predetermined time, and the motor 5 which is an object to be driven is identified by determining whether or not all measured current values are more than a threshold value or equal to or less than the threshold value. In the example shown in FIG. 4, the motor 5A is selected when the current value is more than 875 mA, and the motor 5B is selected when the current value is equal to or smaller than 875 mA. By repeatedly performing the current measurement plural times within a predetermined time, the motor identification can be performed within a short time, and the reliability of the identification of the motor 5 (blood pump 3) can be also enhanced.

The threshold value is set by taking into account coil impedance which is an object to be measured and irregularities in a current value caused by an influence of surface temperatures of the motors at the time of driving the motors. A resistance of the coil changes corresponding to a change in temperature, and a current value changes corresponding to the change in the resistance of the coil. A static temperature of the blood pump is a human body temperature, and a surface temperature of the blood pump may be further increased at the time of driving the blood pump. In this embodiment, a set temperature falls within a range of from 0° C. to 120° C. and hence, the threshold value has a sufficient tolerance with respect to an actual use. Accordingly, it is possible to perform motor identification which matches with actual driving of the motor by setting the threshold value including an influence of a change in temperature. In a vacuum pump of a medical aspirator, a static temperature of a motor is a room temperature, and a surface temperature of the motor is increased at the time of driving the motor and hence, the threshold value may be set by taking into an amount of increased temperature.

Further, in the method of identifying a motor of the blood pump 3, among a plurality of control parameters, the control parameter which matches the identified motor is selected. As main factors relating to the motor specification, the control parameters include the number of magnetic poles, coil impedance and inductance. By selecting the control parameter of the motor 5 which is the object to be driven thus deciding the drive condition such as an applied voltage to the motor 5, a frequency of a voltage pulse by a software of the blood pump control part 6, the occurrence of a human error in the steps ranging from the identification of the motor 5 to the motor starting and steady-state driving can be eliminated.

Further, in the method of driving a motor of the blood pump 3 at the time of starting driving of the motor, the step of identifying the motor 5 which is an object to be driven by the method of identifying a motor of the blood pump 3 which is the previously-mentioned medical pump; the step of selecting a control parameter which matches the identified motor 5; the step of performing magnetic pole alignment between the rotor and the stator by applying a voltage to the motor 5 for a predetermined time; the step of constantly increasing a rotational speed of the motor 5 by applying a motor start voltage pulse to the motor 5 for a predetermined time; and the step of driving the motor 5 at a rotational speed of a steady-state driving of the blood pump 3 are autonomously switched in accordance with a sequence programmed in the blood pump controller 1 which forms the controller.

In such a method of driving a motor of the blood pump 3, a series of steps ranging including: the step of identifying the motor 5; the step of selecting the parameter; the step of performing magnetic pole alignment; and the step of constantly increasing a rotational speed of the motor to a rotational speed for steady-state driving and maintaining the rotational speed for steady-state driving when the rotational speed of the motor becomes a rotational speed for steady-state driving are automatically sequentially switched in accordance with a sequence. Accordingly, a human error can be eliminated by eliminating a human operation and a human decision ranging from the identification of the motor to the steady state driving.

Further, a drive control of the motor 5 is performed by a PWM control. A voltage pulse applied to the motor 5 is switched to a duty of a voltage pulse in magnetic pole alignment, a duty of a motor start voltage pulse, a duty of steady-state driving pulse sequentially after a lapse of a predetermined time.

In starting driving of the motor 5, the motor 5 is controlled by performing the magnetic pole alignment between the rotor and the stator (coils) such that the motor 5 does not step out at the time of starting driving of the motor. Since the motor is not rotated in the magnetic pole alignment, no restriction is imposed on a voltage pulse relating to a drive torque. A rotational load applied to a pump portion 4 (impeller) of the blood pump 3 is large at the time of starting driving of the motor and hence, a drive torque is increased. During a time period from a point of time that the motor is started to steady-state driving, a rotational speed is increased at a predetermined rate by driving in a forced commutation mode. In steady-state driving, a drive torque is set to a value which enables the stable rotation of the motor. A torque during a steady-state driving time may be set smaller than a torque at the time of starting driving of the motor. In this manner, by setting an appropriate duty ratio in the respective drive regions, it is possible to start driving of the motor within a short time while suppressing a consumed current and to bring the motor in a stable driving state with a predetermined rotational speed.

In the blood pump 3 retained in the living body, the motor is required to start driving and to be shifted to steady-state driving within a short time. As described previously, according to the example of the present invention, it is possible to perform shifting the step from the motor identification to the steady-state driving within 4 seconds.

The blood pump controller 1 controls the motor 5 of the blood pump 3 which forms the above-mentioned medical pump. The blood pump controller 3 includes: the switching circuit part 7 configured to apply a direct current voltage or an alternating current voltage in accordance with a predetermined order to the respective coils of three phases, that is, a U phase, a V phase and a W phase; the current detection circuit part 8 configured to measure an electric current which flows into coils of any two phases among the coils of the three phases consisting of the U phase, the V phase and the W phase; and the blood pump control part 6 which performs a control of the current comparison determination part 9 configured to determine the motor 5 which is the object to be driven by comparing a measured current value with a threshold value, and the control parameter selection part 10 configured to select a control parameter which matches the motor which is the object to be driven among a plurality of the control parameters preliminary set based on the measured current value.

The blood pump controller 1 identifies the motor 5 which is the object to be driven based on the detected current value, selects the control parameter which matches the motor 5, and drives the motor 5. The blood pump control part 6 performs a control of the entirety of the blood pump 3 and the blood pump controller 1. The switching control circuit part 7 has a function of applying a voltage to either one of or all of the U phase, the V phase and the W phase in set order based on the control parameter, and a function of inputting a motor drive signal to the motor 5. By autonomously sequentially switching the steps ranging from the identification of the motor 5 to the steady-state driving, a human determination action does not exist and hence, a human error can be eliminated. Further, by preparing the control parameters which correspond to plural kinds of motors 5 in the blood pump controller 1, the plural kinds of motors 5 can be identified using one blood pump controller 1, and the controller 1 can drive the motor 5 which is the object to be driven using the control parameter which matches the motor 5 which is the selected object to be driven.

### [Configuration of Ventricular Assist System 30]

FIG. 5 is an explanatory view showing one example of the ventricular assist system 30. The ventricular assist system 30 includes: the blood pump 3 embedded and retained in the living body; artificial blood vessels 31, 32 which connect the blood pump 3 and the heart for supplying blood; and the blood pump controller 1 having a function of controlling the blood pump 3 outside the living body. The blood pump controller 1 and the blood pump 3 are connected to each other through a medical tube 33 which functions as a drive-line. The medical tube 33 is fixed to a transdermal portion by a medical tube fixing jig 34.

An electric signal line (not shown in the drawing) is made to pass through the medical tube 33. The electric signal line is a cable which is connected to the U phase coil, the V phase coil and the W phase coil of the motor 5 which forms the blood pump 3. The electric signal line is electrically connected to the blood pump controller 1 via the connector 2 (see FIG. 1). The display part 20, the lamp 21, the input part 22, the buzzer 23 and the main switch 24 are disposed on a housing of the blood pump controller 1. FIG. 5 shows one example of the arrangement of these constitutional parts, and these constitutional parts are respectively disposed at places which are easily visually recognized or places where the constitutional parts can be easily manipulated. A first battery 15, a second battery 16 and an emergency battery 17 are housed in the housing (see FIG. 1).

According to the ventricular assist system 30 having such a configuration, when the blood pump controller 1 is started, the steps are autonomously shifted from the identification and starting of the motor 5 (blood pump 3) retained in the living body to steady-state driving of the motor 5 and hence, the occurrence of a human error can be eliminated. Further, in the case where two kinds of motors 5 (blood pumps 3) are used, for example, by connecting and starting one set of blood pump controller 1 having the control parameters which correspond to the plurality of motor specifications, the motor 5 (blood pump 3) retained in the living body can be automatically identified. Accordingly, the blood pump 3 can be started with the control parameter which matches the blood pump 3, and stable driving of the motor 5 can be continued.

The present invention is not limited to the above-mentioned embodiment, and modifications and improvements which can be achieved within the object of the present invention are embraced by the present invention.

For example, in the above-mentioned embodiment, as the specific example, the example is exemplified with respect to the case where two kinds of motors 5 are used. However, the number of kinds of motors 5 is not limited to two, and the present invention is also applicable to the case where the number of kinds of motors 5 is more than two such as three or four. For example, the case may be considered where threshold values of an electric current are set in a stepwise manner, and control parameters which correspond to five kinds of motors 5 are set in the blood pump controller 1. In this case, plural kinds of motors can be controlled using one blood pump controller 1.

1. A method of identifying a motor of a medical pump, the motor of the medical pump being a motor of a three-phase Y-connection formed of coils of three phases consisting of a U phase coil, a V phase coil and a W phase coil, the method comprising the steps of:
detecting a current value of an electric current between the coils of two phases by applying a direct current voltage or an alternating current voltage to the coils of any two phases among the coils of the three phases of the motor which is an object to be driven; and
identifying the motor which is the object to be driven by determining whether the current value which is detected is more than a threshold value which is preliminarily set or equal to or less than the threshold value.

2. The method of identifying a motor of a medical pump according to claim 1, wherein detection of the current value is intermittently performed plural times within a predetermined time, and the motor which is the object to be driven is identified by determining whether all measured current values are more than the threshold value or equal to or less than the threshold value.

3. The method of identifying a motor of a medical pump according to claim 1, wherein the threshold value is set by taking into account irregularities of a current value attributed to an influence of a coil impedance which is an object to be measured or a surface temperature of the motor during driving.

4. The method of identifying a motor of a medical pump according to claim 1, wherein a control parameter which matches the motor which is identified among a plurality of the control parameters is selected.

5. A method of driving a motor of a medical pump comprising the steps of:
identifying the motor which is an object to be driven by the method of identifying a motor of a medical pump according to claim 1;
selecting a control parameter which matches the identified motor;
performing magnetic pole alignment between a rotor and a stator by applying a voltage to the motor for a predetermined time;
constantly increasing a rotational speed of the motor by applying a motor start pulse to the motor for a predetermined time; and
driving the motor at a rotational speed of a steady-state driving of the medical pump, wherein
the steps are autonomously switched in accordance with a sequence programmed in the controller.

6. The method of driving a motor of a medical pump according to claim 5, wherein
a drive control of the motor is performed by a PWM control, and
a voltage pulse applied to the motor is switched to a duty of the voltage pulse at the magnetic pole alignment, a duty of a motor start voltage pulse, and a duty of a steady-state driving voltage pulse sequentially after a lapse of a predetermined time.

7. A controller for controlling the motor of the medical pump described in claim 1, the controller comprising:
a switching circuit part configured to apply a direct current voltage or an alternating current voltage in accordance with a predetermined order to the respective coils of the three phases; and
a control part configured to control: a current detection circuit part configured to measure an electric current which flows into coils of any two phases among the coils of the three phases;
a current comparison determination part configured to determine the motor which is the object to be driven by comparing a measured current value with a threshold value; and a control parameter selection part configured to select a control parameter which matches the motor which is the object to be driven among a plurality of the control parameters preliminary set based on the measured current value.

8. A ventricular assist system comprising:
the medical pump described in claim 1 embedded and retained in a living body; and
a controller disposed outside the living body and connected to the medical pump through a medical tube, wherein the controller comprises
a switching circuit part configured to apply a direct current voltage or an alternating current voltage in accordance with a predetermined order to the respective coils of the three phases; and
a control part configured to control: a current detection circuit part configured to measure an electric current which flows into coils of any two phases among the coils of the three phases; a current comparison determination part configured to determine the motor which is the object to be driven by comparing a measured current value with a threshold value; and a control parameter selection part configured to select a control parameter which matches the motor which is the object to be driven among a plurality of the control parameters preliminary set based on the measured current value.
